(19) United States
(12) Patent Application Publication
(10) Pub. No.: US 2020/0093785 A1
(43) Pub. Date: Mar. 26, 2020

(54) SKIN CARE COMPOSITIONS AND METHODS

(71) Applicant: Deidra Stauff, Pacific Palisades, CA (US)

(72) Inventor: Tamara Stauff, Pacific Palisades, CA (US)

(21) Appl. No.: 16/494,654

(22) PCT Filed: Mar. 22, 2018
(86) PCT No.: PCT/US2018/023870

§ 371 Date: Sep. 16, 2019

Related U.S. Application Data
(60) Provisional application No. 62/476,558, filed on Mar. 24, 2017.

(51) Int. Cl. A61K 31/352; A61K 31/05; A61K 45/06; A61K 9/00; A61P 17/02; A61P 17/14; A61Q 19/08

(52) U.S. Cl. CPC A61K 31/352; A61K 31/05; A61K 45/06; A61Q 19/08; A61K 9/0019; A61P 17/02; A61P 17/14; A61K 9/0014

(57) ABSTRACT

Disclosed herein are compositions and methods for improving the appearance of the skin or for treating disorders of the skin incorporating extracts of plants of the genus Cannabis, and especially incorporating cannabidiolic acid. Provided herein also are methods of using the disclosed compositions for the treatment of skin disorders or improving the appearance of the skin in human and non-human subjects.

### CROSS REFERENCE TO RELATED APPLICATIONS

This application claims the benefit of U.S. Provisional Application Ser. No. 62/476,558 filed Mar. 24, 2017, which is hereby expressly incorporated by reference in its entirety.

### BACKGROUND OF THE INVENTION

### Field

The present disclosure relates generally to methods and compositions for the treatment of the skin, and methods for the manufacture thereof, for such purposes as reducing scarring, reducing wrinkles, and smoothing of lesions related to acne, atopic dermatitis, or other conditions affecting the appearance and health of the skin.

### Background

Numerous compositions and methods have been developed for improving the health and appearance of the skin. Exemplary compositions include cleansers, moisturizers, exfoliants, and cosmetics, while exemplary methods include liposuction, collagen injection, lipoinjection, subcutaneous administration of paralytic toxins, and removal of outer layers of skin by dermabrasion, chemical, or laser peels.

Various compositions incorporating cannabis extracts and cannabinoid preparations have also been disclosed. These preparations are often disclosed to have neurotrophic effects due to the presence of cannabinoids such as tetrahydrocannabinol (THC), and cannabidiol (CBD), each having its own distinctive effect. It is well known in the art that extracts of the cannabis plant must be heated in order to effect the conversion of tetrahydrocannabinolic Acid and cannabadiolic acid to THC and CBD, which are currently recognized as being the active ingredients of existing preparations. Topical preparations of THC and CBD have been proposed as anesthetics or as modes of administration of THC and CBD in order to access the neurotrophic effect of these compounds.

### SUMMARY

The present disclosure relates to compositions comprising an effective amount of cannabadiolic acid and/or tetrahydrocannabinolic acid and further comprising one or more pharmaceutically acceptable excipients. Said excipients may comprise a carrier, diluent, binder, thickener, emulsifier, stabilizer, emollient, abrasive, oil, wax, colloid, humectant, or moisturizer. The compositions of the present disclosure may further comprise one or more additional therapeutic agents. Suitable additional therapeutic agents may comprise one or more of a vitamin, an analgesic, an antipyretic, a cooling agent, a warming agent, a collagenase, a sunscreen, an anesthetic, an antibiotic, an antiviral, an antifungal, a depilatory, a hair-growth enhancer, a steroid, and an antipruritic.

The compositions of the present disclosure may be formulated by any means known in the art, but preferably incorporate an ingredient produced by the steps of: extracting from the plant matter of a plant of the genus Cannabis said tetrahydrocannabinolic Acid and/or cannabadiolic acid utilizing a carrier selected from the group consisting of a liquid C1-C8 alkane, liquid helium, liquid hydrogen, liquid neon, liquid xenon, liquid argon, liquid carbon dioxide, liquid nitrogen, liquid oxygen or any combination of the aforementioned carriers; and removing the carrier; wherein the temperature of the composition does not rise above 37° C. prior to the addition of any excipient or additive. The compositions of the present disclosure may also be made by the aforementioned process wherein the temperature of the composition does not rise above does not rise above 25° C., 20° C., 15° C., 10° C., 5° C., or 0° C. prior to the addition of any excipient or additive.

The plant matter used in the aforementioned process may comprise any part of a plant of the genus Cannabis, including but not limited to the stems, arils, roots, leaves, inflorescences, or seeds. The plant matter may be derived from the root ball of said plant.

The compositions of the present disclosure may be incorporated into a poultice for the treatment of burns, an anti-itch cream, an antibiotic ointment, an after-sun gel, an after-sun lotion, shaving product, deodorant, odorant, insect repellant, facial care products, body care product, cosmetic, soap product, lip product, a shaving cream, a shaving lotion, an after-shave lotion, a roll-on deodorant, a spray deodorant, an air freshener, a room deodorizer, a perfume, a cologne, a hand-soap, a facial soap, a lipstick, a lip balm, a lip gloss, a body lotion, or a shower gel.

Provided herein also is a method of treating wrinkles, scars, atopic dermatitis, moles, skin discolorations, rosacea, insect bites, insect stings, allergenic effects, burns, scrapes, cuts, abrasions, psoriasis, dandruff, pruritus, itching, nasal complaints, sore throats, upper respiratory ailments, acne, athlete's foot, or skin irritation as a result of contact with poison ivy, poison oak, poison sumac, stinging nettle or other poisonous plants comprising identifying a subject in need thereof, and further comprising applying a composition comprising administering an effective amount of the composition of claim 1 to said subject.

The compositions of the present disclosure may be formulated for use in humans, or for veterinary use in a domestic animal, such as a dog, cat, hamster, gerbil, guinea pig, ferret, horse, donkey, mule, cow, domestic buffalo, camel, llama, alpaca, bison, yak, goat, sheep, pig, elk, deer, domestic antelope, or a non-human primate.

The methods of the present disclosure may incorporate topical, subcutaneous, or transdermal administration of the compositions described herein. Subcutaneous administration may utilize injection, or may proceed using a patch, poultice, microneedle, or iontophoresis.

### DETAILED DESCRIPTION

The term “mammal” is used in its usual biological sense. Thus, it specifically includes humans and non-human mammals such as dogs, cats, hamsters, gerbils, guinea pigs, ferrets, horses, donkeys, mules, cows, domestic buffaloes, camels, llamas, alpacas, bison, yaks, goats, sheep, pigs, elk, deer, domestic antelopes, and non-human primates as well as many other species.

“Subject” as used herein, means a human or a non-human animal including but not limited to a dog, cat, hamster, gerbil, guinea pig, ferret, horse, donkey, nude, cow, domestic buffalo, camel, llama, alpaca, bison, yak, goat, sheep, pig, elk, deer, domestic antelope, or a non-human primate selected for treatment or therapy.

“Subject suspected of having” means a subject exhibiting one or more clinical indicators of a disease or condition. In certain embodiments, the disease or condition is dermatitis. In certain embodiments, the disease or condition is atopic dermatitis. In certain embodiments, the disease or condition is stasis dermatitis. In certain embodiments, the disease or condition is toxic dermatitis or contact dermatitis. In certain embodiments, the disease or condition is psoriasis. In certain embodiments, the disease or condition is rosacea. In certain embodiments, the disease or condition is pruritis. In certain embodiments, the disease or condition is acne. In certain embodiments, the disease or condition is scarring or keloid formation. In certain embodiments, the disease or condition is zoster. In certain embodiments, the disease or condition is a herpetic lesion. In certain embodiments, the disease or condition is seborrheic dermatitis. In certain embodiments, the disease or condition is impetigo. In certain embodiments, the disease or condition is ichthyosis vulgaris. In certain embodiments, the disease or condition is lichen planus. In certain embodiments, the disease or condition is actinic keratosis. In certain embodiments, the disease or condition comprises insect bites, insect stings, allergenic effects, burns, scrapes, cuts, abrasions, dandruff, athlete's foot, or skin irritation as a result of contact with poison ivy, poison oak, poison sumac, stinging nettle or other poisonous plants.

“Atopic dermatitis” is also referred to as eczema or atopic eczema, and incorporates one or more of the following symptoms: dry skin that forms a rash; scaly, swollen, and red skin; rash on the face, or inside the knees, elbows, or wrists; blisters that ooze; changes in skin color after repeated episodes; thickened, cracked, dry, scaly skin or skin that looks leathery in patches; and severe itchiness (pruritis), especially at night, along with raw, sensitive, swollen skin from scratching. Atopic dermatitis (eczema) signs and symptoms vary widely from person to person and may further include: red to brownish-gray patches, especially on the hands, feet, ankles, wrists, neck, upper chest, eyelids, inside the bend of the elbows and knees, and, in infants, the face and scalp; small, raised bumps, which may leak fluid and crust over when scratched. Atopic dermatitis most often begins before age 5 and may persist into adolescence and adulthood.

“Subject in need thereof” means a subject identified as in need of a therapy or treatment. “Subject in need thereof” further incorporates a subject desiring, or considered to be in need of, a cosmetic or aesthetic effect.

A therapeutic effect relieves, to some extent, one or more of the symptoms of a disease or disorder, and includes curing the disease or disorder. “Curing” means that the symptoms of active disease are eliminated. However, certain long-term or permanent effects of the disease may exist even after a cure is obtained.

A cosmetic effect improves the subjective appearance of an individual. Such effect may include increasing the tightness or hydration of the skin, reduction in discoloration of the skin, reduction of the appearance of scarring or wrinkling, or other such changes as visibly alter the appearance of the skin or visible external structures of the body.

“Treat,” “treatment,” or “treating,” as used herein refers to administering a pharmaceutical composition for prophylactic and/or therapeutic purposes. The term “prophylactic treatment” refers to treating a patient who does not yet have the relevant disease or disorder, but who is susceptible to, or otherwise at risk of, a particular disease or disorder, whereby the treatment reduces the likelihood that the patient will develop the disease or disorder. The term “therapeutic treatment” refers to administering treatment to a patient already having a disease or disorder.

“Preventing” or “prevention” refers to delaying or forestalling the onset, development or progression of a condition or disease for a period of time, including weeks, months, or years.

“Amelioration” means a lessening of severity of at least one indicator of a condition or disease. In certain embodiments, amelioration includes a delay or slowing in the progression of one or more indicators of a condition or disease. The severity of indicators may be determined by subjective or objective measures which are known to those skilled in the art.

“Modulation” means a perturbation of function or activity. In certain embodiments, modulation means an increase in gene expression. In certain embodiments, modulation means a decrease in gene expression. In certain embodiments, modulation means an increase or decrease in total serum levels of a specific protein. In certain embodiments, modulation means an increase or decrease in free serum levels of a specific protein. In certain embodiments, modulation means an increase or decrease in total serum levels of a specific non-protein factor. In certain embodiments, modulation means an increase or decrease in free serum levels of a specific non-protein factor. In certain embodiments, modulation means an increase or decrease in total bioavailability of a specific protein. In certain embodiments, modulation means an increase or decrease in total bioavailability of a specific non-protein factor.

“Administering” means providing a pharmaceutical agent or composition to a subject, and includes, but is not limited to, administering by a medical professional and self-administering.

Administration of the compounds disclosed herein or the pharmaceutically acceptable salts thereof can be via any of the accepted modes of administration for agents that serve similar utilities including, but not limited to, orally, subcutaneously, intravenously, intranasally, topically, transdermally, intraperitoneally, intramuscularly, intrapulmonarilly, vaginally, rectally, or intraocularly. Topical, intradermal, transdermal, and subcutaneous administrations are customary in treating the indications that are the subject of the preferred embodiments.

Administration of the compounds disclosed herein or cosmetically acceptable formulations thereof can be via any of the accepted modes of administration for agents that serve similar utilities including, but not limited to, orally, subcutaneously, intravenously, intranasally, topically, transdermally, intraperitoneally, intramuscularly, intrapulmonarilly, vaginally, rectally, or intraocularly. Topical, intradermal, transdermal, and subcutaneous administrations are customary in treating the indications that are the subject of the preferred embodiments.

“Parenteral administration,” means administration through injection or infusion. Parenteral administration includes, but is not limited to, subcutaneous administration, intravenous administration, intramuscular administration, intraarterial administration, and intracranial administration.

“Subcutaneous administration” means administration immediately below the skin.

“Intravenous administration” means administration into a vein.

“Intraarterial administration” means administration into an artery.

The term “agent” includes any substance, molecule, element, compound, entity, or a combination thereof It includes, but is not limited to, e.g., protein, polypeptide, peptide or mimetic, small organic molecule, polysaccharide, polynucleotide, and the like. It can be a natural product, a synthetic compound, or a chemical compound, or a combination of two or more substances.

“Pharmaceutical agent” means a substance that provides a therapeutic effect when administered to a subject.

“Pharmaceutical composition” means a mixture of substances suitable for administering to an individual that includes a pharmaceutical agent. For example, a pharmaceutical composition may comprise a modified oligonucleotide and a sterile aqueous solution.

“Active pharmaceutical ingredient” means the substance in a pharmaceutical composition that provides a desired effect.

The term “pharmaceutically acceptable salt” refers to salts that retain the biological effectiveness and properties of the compounds with which they are associated and, which are not biologically or otherwise undesirable. In many cases, the compounds herein are capable of forming acid and/or base salts by virtue of the presence of amino and/or carboxyl groups or groups similar thereto. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, metlianesulfbnic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like; particularly preferred are the ammonium, potassium, sodium, calcium and magnesium salts. Organic bases from whith salts can be derived include, for example, primary, secondary, and tertiary amines, substituted amities including naturally occurring substituted amines, cyclic amines, basic ion exchange resins, and the like, specifically such as isopropylamine, trimethylamine, diethylamine, triethylamine, tripropylamine, and ethanolamine. Many such salts are known in the art, as described in WO 87/05297, Johnston et al., published Sep. 11, 1987 (incorporated by reference herein in its entirety).

The compounds useful as described above can be formulated into pharmaceutical compositions for use in treatment of these conditions. Standard pharmaceutical formulation techniques are used, such as those disclosed in Remington's The Science and Practice of Pharmacy, 21st Ed., Lippincott Williams & Wilkins (2005), incorporated herein by reference in its entirety. Accordingly, some embodiments include pharmaceutical compositions comprising: (a) a safe and therapeutically effective amount of a compound described herein, or pharmaceutically acceptable salts thereof; and (b) a pharmaceutically acceptable carrier, diluent, excipient or combination thereof.

The term “pharmaceutically acceptable carrier” or “pharmaceutically acceptable excipient” includes any and all solvents, diluents, emulsifiers, binders, buffers, dispersion media, coatings, antibacterial and antifungal agents, isotonic and absorption delaying agents and the like, or any other such compound as is known by those of skill in the art to be useful in preparing pharmaceutical formulations. The use of such media and agents for pharmaceutically active substances is well known in the art, Except insofar as any conventional media or agent is incompatible with the active ingredient, its use in the therapeutic compositions is contemplated. Supplementary active ingredients can also be incorporated into the compositions. In addition, various adjuvants such as are commonly used in the art may be included. These and other such compounds are described in the literature, e.g., in the Merck Index, Merck & Company, Rahway, N.J. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press.

Some examples of substances, which can serve as pharmaceutically-acceptable carriers or components thereof, are sugars, such as lactose, glucose and sucrose; starches, such as corn starch and potato starch; cellulose and its derivatives, such as sodium carboxymethyl cellulose, ethyl cellulose, and methyl cellulose; powdered tragacanth; malt; gelatin; talc; solid lubricants, such as stearic acid and magnesium stearate; calcium sulfate; vegetable oils, such as peanut oil, cottonseed oil, sesame oil, olive oil, corn oil and oil of theobroma; polyols such as propylene glycol, glycerine, sorbitol, mannitol, and polyethylene glycol; alginic acid; emulsifiers, such as the TWEENS; wetting agents, such as sodium lauryl sulfate; coloring agents; flavoring agents; tableting agents, stabilizers; antioxidants; preservatives; pyrogen-free water; isotonic saline; and phosphate buffer solutions.

The choice of a pharmaceutically-acceptable carrier to be used in conjunction with the subject compound is determined by the way the compound is to be administered.

The compositions described herein are preferably provided in unit dosage form. As used herein, a “unit dosage form” is a composition containing an amount of a compound that is suitable for administration to a subject, in a single dose, according to good medical practice. The preparation of a single or unit dosage form however, does not imply that the dosage form is administered once per day or once per course of therapy. A unit dosage form may comprise a single daily dose or a fractional sub-dose wherein several unit dosage forms are to be administered over the course of a day in order to complete a daily dose. According to the present disclosure, a unit dosage form may be given more or less often that once daily, and may be administered more than once during a course of therapy. Such dosage forms may be administered in any manner consistent with their formulation, including orally, parenterally, and may be administered as an infusion over a period of time (e.g., from about 30 minutes to about 2-6 hours). While single administrations are specifically contemplated, the compositions administered according to the methods described herein may also be administered as a continuous infusion or via an implantable infusion pump.

The methods as described herein may utilize any of a variety of suitable forms for a variety of routes for administration, for example, for oral, nasal, rectal, topical (including transdermal), ocular, intracerebral, intracranial, intrathecal, intra-arterial, intravenous, intramuscular, subcutaneous, or other parenteral routes of administration. The skilled artisan will appreciate that oral and nasal compositions include compositions that are administered by inhalation, and made using available methodologies. Depending upon the particular route of administration desired, a variety of pharmaceutically-acceptable carriers well-known in the art may be used. Pharmaceutically-acceptable carriers include, for example, solid or liquid fillers, diluents, hydrotropes, surface-active agents, and encapsulating substances. Optional pharmaceutically-active materials may be included, which do not substantially interfere with the activity of the compound. The amount of carrier employed in conjunction with the compound is sufficient to provide a practical quantity of material for administration per unit dose of the compound. Techniques and compositions for making dosage forms useful in the methods described herein are described in the following references, all incorporated by reference herein: Modern Pharmaceutics, 4th Ed., Chapters 9 and 10 (Banker & Rhodes, editors, 2002); Lieberman et al., Pharmaceutical Dosage Forms: Tablets (1989); and Ansel, Introduction to Pharmaceutical Dosage Forms 8th Edition (2004).

Encapsulating substances comprise any substance that may form a capsule or barrier surrounding a particle containing some amount or fraction of an active pharmaceutical substance, cosmetic agent, active principle of a personal care product, or other composition of interest. Such encapsulating substances may be employed in the formulation of any dosage form and especially topical, transdermal, or parenteral dosage forms for subcutaneous or transdermal administration. Exemplary encapsulating substances include but are not limited to, fatty acids, phospholipids, polyols, copolymers, oils, and waxes. In some embodiments, the encapsulating substance may comprise palmitic, stearic, linoleic acid or the like, polyethylene glycol, polypropylene glycol, polylactic acid, polyglycolic acid, polylactic-glycolic acid, poly methyl vinyl ether-maleic anhydride, or block copolyomers such as polylactic-co-glycolic acid, or Poloxamer®/Pluronic®-type block copolymers. The encapsulating substance may also or alternatively comprise any oil or wax capable of forming an emulsion such that a substance of interest may be encapsulated within a nanoparticle, microparticle, liposome, or micelle. Contemplated herein are compositions in which one or more active agents are incorporated into the lipid phase or interfacial region of a micelle, liposome, or other emulsion phase.

Encapsulating substances as defined for topical, transdermal, or parenteral dosage forms for subcutaneous or transdermal administration, or for other pharmaceutical dosage forms, may also be utilized when the compositions of the present disclosure are employed as supplements, cosmetics, or personal care products.

Various oral dosage forms can be used, including such solid forms as tablets, capsules, granules and bulk powders. Tablets can be compressed, tablet triturates, enteric-coated, sugar-coated, film-coated, or multiple-compressed, containing suitable binders, lubricants, diluents, disintegrating agents, coloring agents, flavoring agents, flow-inducing agents, and melting agents. Liquid oral dosage forms include aqueous solutions, emulsions, suspensions, solutions and/or suspensions reconstituted from non-effervescent granules, and effervescent preparations reconstituted from effervescent granules, containing suitable solvents, preservatives, emulsifying agents, suspending agents, diluents, sweeteners, melting agents, coloring agents and flavoring agents.

The pharmaceutically-acceptable carriers suitable for the preparation of unit dosage forms for peroral administration is well-known in the art. Tablets typically comprise conventional pharmaceutically-compatible adjuvants as inert diluents, such as calcium carbonate, sodium carbonate, mannitol, lactose and cellulose; binders such as starch, gelatin and sucrose; disintegrants such as starch, alginic acid and croscarmelose; lubricants such as magnesium stearate, stearic acid, microcrystalline cellulose, carboxymethyl cellulose, and talc. Tablets may also comprise solubilizers or emulsifiers, such as poloxamers, cremophor/Kolliphor®/Lutrol®, methylcellulose, hydroxypropylmethylcellulose, or others as are known in the art. Glidants such as silicon dioxide can be used to improve flow characteristics of the powder mixture. Coloring agents, such as the FD&C dyes, can be added for appearance. Sweeteners and flavoring agents, such as aspartame, saccharin, menthol, peppermint, and fruit flavors, are useful adjuvants for chewable tablets. Capsules typically comprise one or more solid diluents disclosed above. The selection of carrier components depends on secondary considerations like taste, cost, and shelf stability, which can be readily made by a person skilled in the art.

Peroral (PO) compositions also include liquid solutions, emulsions, suspensions, and the like. The pharmaceutically-acceptable carriers suitable for preparation of such compositions are well known in the art. Typical components of carriers for syrups, elixirs, emulsions and suspensions include ethanol, glycerol, propylene glycol, polyethylene glycol, liquid sucrose, sorbitol and water. For a suspension, typical suspending agents include methyl cellulose, sodium carboxymethyl cellulose, AVICEL RC-591, tragacanth and sodium alginate; typical wetting agents include lecithin and polysorbate 80; and typical preservatives include methyl paraben and sodium benzoate. Peroral liquid compositions may also contain one or more components such as sweeteners, flavoring agents and colorants disclosed above.

Such compositions may also be coated by conventional methods, typically with pH or time-dependent coatings, such that the subject compound is released in the gastrointestinal tract in the vicinity of the desired topical application, or at various times to extend the desired action. Such dosage forms typically include, but are not limited to, one or more of cellulose acetate phthalate, polyvinylacetate phthalate, hydroxypropyl methyl cellulose phthalate, ethyl cellulose, Eudragit coatings, waxes and shellac.

Compositions described herein may optionally include other drug actives.

Other compositions useful for attaining systemic delivery of the subject compounds include sublingual, buccal and nasal dosage forms. Such compositions typically comprise one or more of soluble filler substances such as sucrose, sorbitol and mannitol; and binders such as acacia, microcrystalline cellulose, carboxymethyl cellulose and hydroxypropyl methyl cellulose. Glidants, lubricants, sweeteners, colorants, antioxidants and flavoring agents disclosed above may also be included.

In some embodiments, the compositions of the present disclosure comprise a liquid composition, which is formulated for topical ophthalmic use. Such a composition is formulated such that it can be administered topically to the eye. The comfort may be maximized as much as possible, although sometimes formulation considerations (e.g. drug stability) may necessitate less than optimal comfort. In the case that comfort cannot be maximized, the liquid may be formulated such that the liquid is tolerable to the patient for topical ophthalmic use. Additionally, an ophthalmically acceptable liquid may either be packaged for single use, or contain a preservative to prevent contamination over multiple uses.

For ophthalmic application, solutions or medicaments are often prepared using a physiological saline solution as a major vehicle. Ophthalmic solutions may preferably be maintained at a comfortable pH with an appropriate buffer system. The formulations may also contain conventional, pharmaceutically acceptable preservatives, stabilizers and surfactants.

Preservatives that may be used in the pharmaceutical compositions disclosed herein include, but are not limited to, benzalkonium chloride, PHMB, chlorobutanol, thimerosal, phenylmercuric, acetate and phenylmercuric nitrate. A useful surfactant is, for example, Tween 80. Likewise, various useful vehicles may be used in the ophthalmic preparations disclosed herein. These vehicles include, but are not limited to, polyvinyl alcohol, povidone, hydroxypropyl methyl cellulose, poloxamers, carboxymethyl cellulose, hydroxyethyl cellulose and purified water.

Tonicity adjustors may be added as needed or convenient. They include, but are not limited to, salts, particularly sodium chloride, potassium chloride, mannitol and glycerin, or any other suitable ophthalmically acceptable tonicity adjustor.

Various buffers and means for adjusting pH may be used so long as the resulting preparation is ophthalmically acceptable. For many compositions, the pH will be between 4 and 9. Accordingly, buffers include acetate buffers, citrate buffers, phosphate buffers and borate buffers. Acids or bases may be used to adjust the pH of these formulations as needed.

Ophthalmically acceptable antioxidants include, but are not limited to, sodium metabisulfite, sodium thiosulfate, acetylcysteine, butylated hydroxyanisole and butylated hydroxytoluene.

Other excipient components, which may be included in the ophthalmic preparations, are chelating agents. A useful chelating agent is disodium EDTA or sodium/calium EDTA, although other chelating agents may also be used in place or in conjunction with it.

For topical use, including for transdermal administration, creams, ointments, gels, solutions or suspensions, etc., containing the compositions disclosed herein are employed. Topical formulations may generally be comprised of a pharmaceutical carrier, co-solvent, emulsifier, penetration enhancer, preservative system, emollient, and optionally, abrasive or filler. Cosmetic preparations may also be comprised of a co-solvent, emulsifier, penetration enhancer, preservative system, emollient, and optionally, abrasive or filler, or any subset or combination thereof.

For intravenous administration, the compounds and compositions described herein may be dissolved or dispersed in a pharmaceutically acceptable diluent, such as a saline or dextrose solution. Suitable excipients may be included to achieve the desired pH, including but not limited to NaOH, sodium carbonate, sodium acetate, HCl, and citric acid. In various embodiments, the pH of the final composition ranges from 2 to 8, or preferably from 4 to 7. Antioxidant excipients may include sodium bisulfite, acetone sodium bisulfite, sodium formaldehyde, sulfoxylate, thiourea, and EDTA. Other non-limiting examples of suitable excipients found in the final intravenous composition may include sodium or potassium phosphates, citric acid, tartaric acid, gelatin, and carbohydrates such as dextrose, mannitol, and dextran. Further acceptable excipients are described in Powell, et al., Compendium of Excipients for Parenteral Formulations, PDA J Pharm Sci and Tech 1998, 52 238-311 and Nema et al., Excipients and Their Role in Approved Injectable Products: Current Usage and Future Directions, PDA J Pharm Sci and Tech 2011, 65 287-332, both of which are incorporated herein by reference in their entirety. Antimicrobial agents may also be included to achieve a bacteriostatic or fungistatic solution, including but not limited to phenylmercuric nitrate, thimerosal, benzethonium chloride, benzalkonium chloride, phenol, cresol, and chlorobutanol.

The compositions for intravenous administration may be provided to caregivers in the form of one more solids that are reconstituted with a suitable diluent such as sterile water, saline or dextrose in water shortly prior to administration, In other embodiments, the compositions are provided in solution ready to administer parenterally. In still other embodiments, the compositions are provided in a solution that is further diluted prior to administration. In embodiments that include administering a combination of a compound described herein and another agent, the combination may be provided to caregivers as a mixture, or the caregivers may mix the two agents prior to administration, or the two agents may be administered separately.

The actual unit dose of the active compounds described herein depends on the specific compound, and on the condition to be treated. In some embodiments, the dose may be from about 0.01 mg/kg to about 120 mg/kg or more of body weight, from about 0.05 mg/kg or less to about 70 mg/kg, from about 0.1 mg/kg to about 50 mg/kg of body weight, from about 1.0 mg/kg to about 10 mg/kg of body weight, from about 5.0 mg/kg to about 10 mg/kg of body weight, or from about 10.0 mg/kg to about 20.0 mg/kg of body weight. In some embodiments, the dose may be less than 100 mg/kg, 90 mg/kg. 80 mg/kg, 70 mg/kg, 60 mg/kg, 50 mg/kg, 40 mg/kg, 30 mg/kg, 25 mg/kg, 20 mg/kg, 10 mg/kg. 7.5 mg/kg. 5 mg/kg, 4 mg/kg, 2.5 mg/kg, 1 mg/kg, 0.5mg/kg, 0.1 mg/kg, or 0.05 mg/kg of body weight. In some embodiments, the actual unit dose is 0.1, 0.3, 1.0, 3.0, or 10.0 mg/kg of body weight. Thus, for administration to a 70 kg person, the dosage range would be from about 0.7 mg to 8400 mg, from about 3.5 mg to about 4900 mg, from about 70 gm to about 700 mg, from about 350 mg to about 700 mg, from about 70 mg to about 1400 mg, from about 17 mg to about 8000 mg, from about 35 mg or less to about 7000 mg or more, from about 70 mg to about 6000 mg, from about 100 mg to about 5000 mg, or from about 200 mg to about 3000 mg. In some embodiments, the actual unit dose is 5 mg. In some embodiments the actual unit dose is 10 mg. In some embodiments, the actual unit dose is 25 mg.

“Mode of administration” as used herein refers to the means by which a compound is administered to a subject. As used herein, “mode of administration” comprises the dosage form (for example, a tablet, powder, dissolved liquid, suspension, emulsion, aerosol, etc.) and mechanism by which the dosage form is applied to the subject (for example, by injection, such as subcutaneously, intramuscularly, intraperitoneally, intravenously, or intraarterially; topically, such as by cream, lotion, or patch; orally, such as by a pill, dissolved liquid, oral suspension, buccal film, or mouthrinse; nasally, such as by a nasal aerosol, powder, or spray; or ocularly, such as by an eye drop). As used herein, “mode of administration” also comprises the dose, dose amount, and dosing schedule by which a compound is administered to a subject.

As used herein, “duration of the treatment” refers to the time commencing with administration of the first dose and concluding with the administration of the final dose, such length of time being determined by one of ordinary skill in the art of treating diseases involving disorders of the skin or administering cosmetic treatments for the improvement of the external appearance of the skin.

As used herein and in the appended claims, the singular forms “a,” “and,” and “the” include plural referents unless the context clearly dictates otherwise. Thus, for example, reference to “a probe” includes a plurality of such cells and reference to “the cell” includes reference to one or more cells and equivalents thereof known to those skilled in the art, and so forth.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

Unless defined otherwise, all technical and scientific terms used herein have the same meaning as commonly understood to one of ordinary skill in the art to which this disclosure belongs. Although any methods and reagents similar or equivalent to those described herein can be used in the practice of the disclosed methods and compositions, the exemplary methods and materials are now described.

In any of the foregoing embodiments, it will be understood by one of ordinary skill in the art that both pharmaceutical (i.e., meant to diagnose, treat or cure a disease or condition) and cosmetic (i.e., meant to affect the subjective appearance of an individual) uses are contemplated.

In some embodiments, the composition comprises one or more active pharmaceutical ingredients. In some further embodiments, the active pharmaceutical ingredient is a cannabidiolic acid or tetrahydrocannabinolic acid. In some further embodiments, the composition further comprises one or more additional active pharmaceutical ingredients, In some further embodiments, the composition comprises one or more of an antibiotic, anti-inflammatory, antipruritic, antifungal, anesthetic, or other active pharmaceutical ingredient.

In some embodiments, the cannabidiolic acid and/or tetrahydrocannabinolic acid are present in a topical composition. Such a topical composition can be in the form of a solid, semi-solid, plaster, solution, suspension, lotion, cream, foam, gel, paste, poultice, emulsion, or a combination thereof.

In some embodiments, a method of treating insect bites, insect stings, allergenic effects, burns, scrapes, cuts, abrasions, psoriasis, dandruff, pruritus, itching, nasal complaints, sore throats, upper respiratory ailments, acne, athlete's foot, or skin irritation as result of contact with poison ivy, poison oak, or poison sumac is provided comprising applying the a composition disclosed herein to a subject in need thereof

In some embodiments, the topical composition can be selected from a poultice for the treatment of burns, an anti-itch cream, an antibiotic ointment, an after-sun gel, or an after-sun lotion.

In some embodiments, the topical composition can be a personal care product selected from the group consisting of shaving products, deodorants, odorants, insect repellents, facial care products, body care products, cosmetics, soap products, and lip products.

In some embodiments, the topical composition can be selected from the group consisting of a shaving cream, a shaving lotion, and after-shave lotion, a roll-on deodorant, a spray deodorant, an air freshener, a room deodorizer, a perfume, a cologne, a hand-soap, a facial soap, a lipstick, a lip balm, a lip gloss, a body lotion, and a shower gel.

In some embodiments, a method for enhancing the massage therapy treatment is provided comprising applying a topical composition disclosed herein to a subject in need thereof. In some embodiments, the topical composition can be an aphrodisiac.

The topical compositions described herein may comprise one or more cooling compounds. The topical composition can be a “pharmaceutical composition” or a composition intended for “personal care.” By “pharmaceutical composition”, it is meant a composition which is used to treat the symptoms and/or root causes of a disease or ailment. In one embodiment, the pharmaceutical composition comprises one or more compounds of the present invention and at least one pharmaceutically acceptable carrier. The pharmaceutical composition category includes both the prescription medications and the over-the-counter medications. The present compound may or may not be the therapeutically active ingredient in the pharmaceutical composition. In general, over the counter (OTC) product and oral hygiene product generally refer to product for household and/or personal use which may be sold without a prescription and/or without a visit to a medical professional. Examples of the OTC products include, but are not limited to skin creams, lip balms, ointments, unguents, poultices, plasters, patches, or the like, as well as moisturizers, acne medications, anti-itch creams, burn ointments, sunscreens, anti-wrinkle creams, skin tighteners, topical analgesics and/or anesthetics. Topical analgesics and/or anesthetics include any topical creams/ointments/gels used to alleviate superficial or deep-seated aches and pains, e.g. muscle pain; teething gel; toothpaste; sports creams; patches with analgesic ingredient; and combinations thereof. In some embodiments, the pharmaceutical composition is hemorrhoid product. In some embodiments, the topical composition can be a sinus relief product or a nasal spray. The topical pharmaceutical composition can further be in the form of a first aid product. For example, the topical composition can be applied as an ointment or on a bandage, band aid, or in a spray.

In some embodiments, the topical composition can be a composition for the treatment and alleviation of pain in a subject. Topical compositions for providing such pain relief can comprise a combination of one or more of cannabidiolic acid or tetrahydrocannabinolic acid as disclosed herein. The topical composition for the relief of pain can further comprise an additional active ingredient. In some embodiments the additional active ingredient can be an anti-inflammatory agent.

In some embodiments, the topical composition can be applied in the treatment of allergenic effects. In some embodiments a topical composition comprising one or more compounds as disclosed herein can further comprise an antibacterial agent, an anti-inflammatory agent, and/or a corticosteroid. In some embodiments, the topical composition can be used in the treatment of insect bites or insect stings. In some embodiments, the topical composition can be used in the treatment of psoriasis or eczema.

In some embodiments, a topical pharmaceutical composition as disclosed herein can be a composition for treatment of skin irritation as a result of contact with poison ivy, poison oak, or poison sumac. For example, the topical pharmaceutical composition can be a composition for removing urushiol, the primary irritant contracted from poison ivy, poison oak, or poison sumac. In some embodiments, the compositions provide a soothing effect to a subject having contracted poison ivy, poison oak, or poison sumac.

As used herein, a “personal care composition” refers to a composition to be directly applied to the skin, mucosa, hair, nails or other surface area of the body. Examples of personal care composition include, but are not limited to, a skincare or haircare composition, such as sunscreen cream, sunburn lotions, shaving cream, plasters, shampoos, conditioners, face cleaners, facial washes, soaps, bath oils or bath foam, antiperspirants, and deodorant; a cosmetic composition, such as moisturizer, lip balms, cosmetic balms, foundation, etc.; an insect repellent composition; or an insecticide composition.

In some embodiments, a topical composition as disclosed herein can be a skincare product. Examples of skincare products include, but are not limited to face washing creams, scar smoothing products, skin irritation soothing products (oils, sprays, salves, lotions, ointments, or gels), facial cleansing wipes, body cleansing wipes, anti-aging facial and/or eye creams, pore cleansing strips, pore cleansing oils, pore cleansing ointments, hair removal products (wax, wax strips, roll-ons, creams, gels, lotions, etc.), varnishing creams, cleansing creams, cold creams, massage creams, milky lotions, skin toning lotion, cosmetic solution, packs, makeup remover, and the like; as makeup cosmetics, foundations, face powders, pressed powders, talcum powders, lip sticks, lip creams, cheek powders, eyeliners, mascara, eye shadows, eyebrow pencils, eye packs, nail enamels, nail enamel removers, and the like.

In some embodiments, a topical composition as disclosed herein can be a body care or bathing composition. For example, the topical composition can be a shaving cream, a shaving gel, a shaving lotion, an after-shave lotion, an after-shave gel, a medicated soap, sanitizing gel, a hand sanitizes, sanitizing wipes, cool nasal tissues (tissues that have lotion on them), sports cooling towels, cooling fabrics, sport shirts, eye pillows, sheets, cooling pillows, hats, diapers, adult diapers, potty-training pants and pull-ups, talcum powder, baby powder, slimming gels, a bath soap, a face soap, a hand soap, exfoliating washes, hand scrub, foot scrub, shower mists, shower sprays, a body washing soap, a body washing gel, a bath salt, a bath tablet, a bath foam, a bubble-bath concentrate, a bath oil, a bath perfume, a bath capsule, a milk bath, a bath gel, or a bath cube.

In some embodiments, a topical composition as disclosed herein can be a composition for the enhancement of massage therapy treatment. For example, the topical composition can be in the form of a massage oil, massage cream, massage lotion, or massage gel that comprises cannabidiolic acid and/or tetrahydrocannabinolic acid.

In one embodiment, the composition disclosed herein comprises i) compounds as disclosed and described herein, individually or in combination; ii) a carrier; and iii) optionally at least one adjuvant. The term “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. In addition, various adjuvants such as are commonly used in the art may be included. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press, which is incorporated herein by reference in its entirety.

The term “adjuvant” denotes an additive which supplements, stabilizes, maintains, or enhances the intended function or effectiveness of the active ingredient, such as the compound of the present invention. In one embodiment, the at least one adjuvant comprises one or more topically acceptable materials that may be used to preserve or alter the properties of the topical composition disclosed herein. These materials include, but are not limited to, materials having anti-acne, anti-ageing, anti-wrinkle, antifungal, anti-inflammatory, antimicrobial, antioxidant, antiperspirant, antidandruff, anti-dermatitis, antipruritic, anti-emetic, anti-dry skin, anti-psoriatic, anti-seborrhea, anti-asthmatic, astringents, bronchodilators, biocides, chemical exfoliants, cleansers, colorants, corticosteroids, deodorants, depigmenting, depilating, emollients, epilating, analgesics, hair conditioners, hormones, humectants, light-interacting, luster-imparting, make-up removers, pH adjusters, powders, rheological modifiers, shine-imparting, skin bleaching, skin conditioning, skin protecting, tanning, UV screening vitamins, and/or wound-healing properties.

In one embodiment, a topical formulation disclosed herein can be in a form selected from the group consisting of liquid, including solution and suspension, solid, foamy material, emulsion, paste, gel, cream, and a combination thereof, such as a liquid containing a certain amount of solid contents. In one embodiment, the flavoring concentrate formulation is in form of a liquid including aqueous-based and nonaqueous-based.

In some embodiments, the topical composition is an ophthalmic composition. A liquid composition, which is formulated for topical ophthalmic use, is formulated such that it can be administered topically to the eye. The comfort may be maximized as much as possible, although sometimes formulation considerations (e.g. compound stability) may necessitate less than optimal comfort. In the case that comfort cannot be maximized, the liquid may be formulated such that the liquid is tolerable to the patient for topical ophthalmic use. Additionally, an ophthalmically acceptable liquid may either be packaged for single use, or contain a preservative to prevent contamination over multiple uses.

For ophthalmic application, solutions or medicaments are often prepared using a physiological saline solution as a major vehicle. Ophthalmic solutions may preferably be maintained at a comfortable pH with an appropriate buffer system. The formulations may also contain conventional, biologically acceptable preservatives, stabilizers and surfactants.

Preservatives that may be used in the topical compositions disclosed herein include, but are not limited to, benzalkonium chloride, PHMB, chlorobutanol, thimerosal, phenylmercuric, acetate and phenylmercuric nitrate. A useful surfactant is, for example, Tween 80. Likewise, various useful vehicles may be used in the ophthalmic preparations disclosed herein, These vehicles include, but are not limited to, polyvinyl alcohol, povidone, hydroxypropyl methyl cellulose, poloxamers, carboxymethyl cellulose, hydroxyethyl cellulose and purified water.

Tonicity adjustors may be added as needed or convenient. They include, but are not limited to, salts, particularly sodium chloride, potassium chloride, mannitol and glycerin, or any other suitable ophthalmically acceptable tonicity adjustor.

Various buffers and means for adjusting pH may be used so long as the resulting preparation is ophthalmically acceptable. For many compositions, the pH will be between 4 and 9. Accordingly, buffers include acetate buffers, citrate buffers, phosphate buffers and borate buffers. Acids or bases may be used to adjust the pH of these formulations as needed.

Ophthalmically acceptable antioxidants include, but are not limited to, sodium metabisulfite, sodium thiosulfate, acetylcysteine, butylated hydroxyanisole and butylated hydroxytoluene.

Other excipient components, which may be included in the ophthalmic preparations, are chelating agents. A useful chelating agent is edetate disodium, although other chelating agents may also be used in place or in conjunction with it.

Topical formulations may generally be comprised of a pharmaceutical carrier, co-solvent, emulsifier, penetration enhancer, preservative system, and emollient.

In some embodiments, a topical composition as disclosed herein may comprise an aqueous component. For example, the composition can be a cream, lotion, ointment, conditioning shampoo, moisturizing hand soap, or other composition for administration to the skin or external structures of the body. In some embodiments, the topical composition disclosed herein can comprise about 35% (w/w) to about 90% (w/w), about 40% (w/w) to about 85% (w/w), about 45% (w/w) to about 80% (w/w), about 50% (w/w) to about 75% (w/w), about 55% (w/w) to about 70% (w/w), about 60% (w/w) to about 65% w/w), or about 62% (w/w) of water. In some embodiments, the topical composition disclosed herein can comprise at least 50% (w/w), 55% (w/w), 60% (w/w), 65% (w/w), 70% (w/w), 75% (w/w), 80% (w/w), or 85% (w/w) of water. In some embodiments, the topical composition disclosed herein can comprise up to 50% (w/w), 55% (w/w), 60% (w/w), 65% (w/w), 70% (w/w), 75% (w/w), 80% (w/w), or 85% (w/w) of water. In some embodiments, the topical composition disclosed herein can comprise 50% (w/w), 55% (w/w), 60% (w/w), 65% (w/w), 70% (w/w), 75% (w/w), 80% (w/w), or 85% (w/w) of water or a range defined by any two of the preceding values.

Some embodiments provide a topical composition including skin penetration enhancers. Examples of suitable skin penetration enhancers include alcohols, fatty acids, fatty acid esters, polyols, sulphoxides, glyceryl monooleate, lauryl lactate, Dodecyl-2-(N,N-dimethyl)-amino propionate (DDAIP), N-(4-bromobenzoyl)-S,S-ditnethyliminosulfurane, NexACT enhancers, 2-nonyl-1,3-dioxolane (SEPA®), 1-dodecylazacycloheptan-2-one (Axone®), pyrrolidones, essential oil, terpenes, terpenoids, oxazolidinones, urea and the like. Other examples of suitable skin penetration enhancers and a description of their mechanism of action may be found in Goodman and Barry, “Percutaneous Absorption,” in Mechanisms-Methodology-Drug Delivery, 2nd Edition, Bronaugh and Maibach, eds., 1989, pp. 567-593, Marcel Dekker, Inc., NY, which is incorporated herein by reference in its entirety.

In some embodiments, the skin penetration enhancer can be selected from the group consisting of n-octanol, D-limonene, oleic acid, cineol, isopropyl myristate, monooleate, monoolein, sodium oleate, oleyl oleate, laurylcapram, sodium lauryl sulfate, bisabolol, lauric acid, myristic acid, isopropyl palmitate, diisopropyl adipate, dimethyl isosorbide, propylene glycol, butylene glycol, polyethylene glycol, dipropylene glycol, ethoxydiglycol, and pentylene glycol or combinations thereof.

In some embodiments, skin penetration can be achieved by formulating the compositions disclosed herein into a nanoparticle or nanoemulsion. Such particles and emulsions are known in the art and include but are not limited to, polylactic acid particles, polylactic/glycolic acid nanoparticles, polystyrene nanoparticles, silicon dioxide nanoparticles, metallic nanoparticles, water-in-oil emulsions, oil-in-water emulsions, polymer nanoparticles and emulsions, and block copolymer nanoparticles and emulsions.

In some embodiments, the topical composition disclosed herein can comprise at least 5% (w/w), 6% (w/w), 7% (w/w), 8% (w/w), 9% (w/w), 10% (w/w), 11% (w/w), or 12% (w/w) of a skin penetration enhancer. In some embodiments, the topical composition disclosed herein can comprise 5% (w/w), 6% (w/w), 7% (w/w), 8% (w/w), 9% (w/w), 10% (w/w), 11% (w/w), 12% (w/w), 13% (w/w), 14% (w/w), 15% (w/w), 16% (w/w), 17% (w/w), 18% (w/w), 19% (w/w), 20% (w/w), 21% (w/w), 22% (w/w), 23% (w/w), 24% (w/w), 25% (w/w), 26% (w/w), 27% (w/w), 28% (w/w), 29% (w/w), or 30% (w/w)) of a skin penetration enhancer or a range defined by any two of the preceding values.

In some embodiments, a topical composition disclosed herein can comprise at least 1% (w/w), 2% (w/w), 3% (w/w), 4% (w/w), 5% (w/w), 6% (w/w), 7% (w/w), 8% (w/w), 9% (w/w), 10% (w/w), 11% (w/w), or 12% (w/w) of an emollient. In some embodiments, the topical composition disclosed herein can comprise 1% (w/w), 2% (w/w), 3% (w/w), 4% (w/w), 5% w/w), 6% (w/w), 7% (w/w), 8% (w/w), 9% (w/w), 10% (w/w), 11% (w/w), 12% (w/w), 13% (w/w), 14% (w/w), 15% (w/w), 16% (w/w), 17% (w/w), 18% (w/w), 19% (w/w), 20% (w/w), 21% (w/w), 22% (w/w), 23% (w/w), 24% (w/w), 25% (w/w), 26% (w/w), 27% (w/w), 28% (w/w), 29% (w/w), or 30% (w/w)) of an emollient or a range defined by any two of the preceding values.

In some embodiments, a topical composition as disclosed herein can comprise natural fats and oils. In some embodiments, the natural fats and oils can be selected from the group consisting of citrus oil, olive oil, avocado oil, apricot oil, babassu oil, borage oil, camellia oil, canola oil, castor oil, coconut oil, corn oil, cottonseed oil, emu oil, evening primrose oil, hydrogenated cottonseed oil, hydrogenated palm kernel oil, maleated soybean oil, meadowfoam oil, palm kernel oil, peanut oil, rapeseed oil, grapeseed oil, safflower oil, sphingolipids, seed almond oil, tall oil, lauric acid, palmitic acid, stearic acid, linoleic acid, stearyl alcohol, lauryl alcohol, myristyl alcohol, behenyl alcohol, rose hip oil, calendula oil, chamomile oil, eucalyptus oil, juniper oil, sandlewood oil, tea tree oil, cocoa butter, jojoba oil, shea butter, lanolin, animal fat, paraffin, beeswax, calendula oil, sunflower oil, and soybean oil, or combinations thereof.

In some embodiments, a topical composition disclosed herein can comprise at least 5% (w/w), 6% (w/w), 7% (w/w), 8% (w/w), 9% (w/w), 10% (w/w) 11% (w/w), or 12% (w/w) of a vegetable oil. In some embodiments, the topical composition disclosed herein can comprise 5% (w/w), 6% (w/w), 7% (w/w), 8% (w/w), 9% (w/w), 10% (w/w), 11% (w/w), 12% (w/w), 13% (w/w), 14% (w/w), 15% (w/w), 16% (w/w), 17% (w/w), 18% (w/w), 19% (w/w), 20% (w/w), 21% (w/w), 22% (w/w), 23% (w/w), 24% (w/w), 25% (w/w), 26% (w/w), 27% (w/w), 28% (w/w), 29% (w/w), or 30% (w/w)) of a vegetable oil or a range defined by any two of the preceding values. In a typical embodiment, the topical composition disclosed herein can comprise coconut oil.

In some embodiments, a topical composition as disclosed herein may optionally further comprise ingredients to relieve irritation, such as anti-itch agents. In some embodiments, the anti-itch agents may be present in the topical composition disclosed herein in an amount of from about 0.1% to about 33% (w/w), more typically, from about 0.5% to about 5% (w/w). Examples of suitable anti-itch agents are listed below, as well as the preferred concentration for each agent, given in percent by weight of the composition: lauromacrogols, benzocaine (about 5% to about 20%), butamben picrate (about 1%), dibucaine (about 0.25% to about 1%), dibucaine hydrochloric acid (0.25% to about 1%), dimethisoquin hydrochloric acid (about 0.3% to about 0.5%), dyclonine hydrochloric acid (about 0.5% to about 1%), lidocaine (about 0.5% to about 5%), lidocaine hydrochloric acid (about 0.5% to about 5%), pramoxine hydrochloric acid (about 0.5% to about 1%), tetracaine (about 1% to about 2%), tetracaine hydrochloric acid (about 1% to about 2%), benzyl alcohol (about 10% to about 33%), camphor (about 0.1% to about 3%), juniper tar (about 1% to about 5%), menthol (about 0.1% to about 1%), phenol (about 0.5% to about 1.5%), phenolate sodium (about 0.5% to about 1.5%), resorcinol (about 0.5% to about 3%), diphenhydramine hydrochloric acid (about 1% to about 2%), tripelennamine hydrochloric acid (about 0.5% to about 2%), hydrocortisone (about 0.1% to about 5%, preferably about 0.5% to about 2.5%), and combinations thereof. In some embodiments, the topical composition disclosed herein may optionally also comprise cosmetic anti-itch ingredients such as, for example, Symcalmin® (Symrise GmbH & Co., Holzminden, Germany).

In some embodiments, the compositions may include adjunct components conventionally found in pharmaceutical compositions in their art-established fashion and at their art-established levels. For example, the compositions may comprise additional compatible pharmaceutically active materials for combination therapy, such as antimicrobials, antioxidants, anti-parasitic agents, antipruritics, antifungals, antiseptic actives, biological actives, astringents, keratolytic actives, local anaesthetics, anti-stinging agents, anti-reddening agents, skin soothing agents, and combinations thereof.

In some embodiments, the compositions may include colorants, deodorants, fragrances, perfumes, emulsifiers, anti-foaming agents, lubricants, natural moisturizing agents, skin conditioning agents, skin protectants and skin benefit agents (e.g., aloe vera and laponite), solvents, solubilizing agents, suspending agents, wetting agents, humectants, preservatives, propellants, dyes and/or pigments, and combinations thereof. In some embodiments, the compositions may have a particularly pleasant fragrance. In some embodiments, the compositions may have a particularly pleasant texture. In some embodiments, the compositions may have a particularly pleasant soothing effect.

In some embodiments, the compositions may include excipients conventionally found in topical compositions,

In some embodiments, the excipients can include a viscosity-adjusting agent. In sonic embodiments, the viscosity adjusting agents can be selected from the group consisting of long chain alcohols, cellulose ethers, gums, magnesium aluminum silicate, silica, microcrystalline wax, beeswax, paraffin, and cetyl palmitate, homopolymers, and copolymers. In some embodiments, the long chain alcohols can be cetyl alcohol, stearyl alcohol, or cetearyl alcohol. In some embodiments, the cellulose ethers can be hydroxypropylmethylcellulose, hydroxy ethylcellulose, hydroxypropylcellulose, or carboxymethylcellulose. In some embodiments, the gum can be xanthan gum or sclerotium gum. In a particular embodiment, the viscosity adjusting agents can include xanthan gum. In another embodiment, the viscosity adjusting agents is xanthan gum. In another embodiment, the viscosity adjusting agent is a polyalkylene oxide such as polyethylene glycol. In other embodiments, the viscosity adjusting agent is pullulan. In other embodiments, the viscos adjusting agent is a polyvinyl halide, such as polyvinyl chloride.

In some embodiments, a topical composition disclosed herein can comprise at least 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1.0% (w/w), 1.1% (w/w), or 1.2% (w/w) of a viscosity adjusting agent. In some embodiments, the topical composition disclosed herein can comprise 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1.0% (w/w), 1.1% (w/w), 1.2% (w/w), 1.5% (w/w), 2% (w/w), 3% (w/w), 4% (w/w), 5% (w/w), 6% (w/w), 7% (w/w), 8% (w/w), 9% (w/w), 10% (w/w), 20% (w/w) or 30% (w/w) of a viscosity adjusting agent or a range defined by any two of the preceding values.

In some embodiments, a topical composition disclosed herein can comprise at least 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1.0% (w/w), 1.1% (w/w), or 1.2% (w/w) of xanthan gum. In some embodiments, the topical composition disclosed herein can comprise 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1.0% (w/w), 1.1% (w/w), 1.2% (w/w), 1.5% (w/w), 2% (w/w), 3% (w/w), 4% (w/w), 5% (w/w), 6% (w/w), 7% (w/w), 8% (w/w), 9% (w/w), 10% (w/w), 20% (w/w) or 30% (w/w) of xanthan gum or a range defined by any two of the preceding values.

In some embodiments, the excipients can include a topical pharmaceutical and cosmetically-acceptable emollient. As used herein, “emollients” refer to materials used for the prevention or relief of dryness, as well as for the protection of the skin. Sagarin, Cosmetics, Science and Technology, 2nd Edition, Vol. 1, pp. 32-43 (1972), which is incorporated herein by reference in its entirety, contains numerous examples of suitable materials for use as emollients. Examples of classes of useful emollients include, but are not limited to, hydrocarbon oils and waxes such as mineral oil, petrolatum, paraffin, ceresin, ozokerite, microcrystalline wax, polyethylene, and perhydrosqualene; silicone oil, such as dimethyl polysiloxanes, methylphenyl polysiloxanes, and water-soluble and alcohol-soluble silicone glycol copolymers. Other suitable emollients include triglyceride esters such as vegetable and animal fats and oils including castor oil, safflower oil, cotton seed oil, corn oil, olive oil, cod liver oil, almond oil, avocado oil, palm oil, lanolin and derivatives. In a typical embodiment, the emollient can include glycerol.

In some embodiments, a topical composition disclosed herein can comprise at least 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1.0% (w/w), 1.1% (w/w), 1.2% (w/w), 1.3% (w/w), 1.4% (w/w), 1.5% (w/w), 1.6% (w/w), 1.7% (w/w), 1.8% (w/w), 1.9% (w/w), 2.0% (w/w), 2.1% (w/w), 2.2% (w/w), 2.3% (w/w), 2.4% (w/w), 2.5% (w/w), 2.6% (w/w), 2.7% (w/w), 2.8% (w/w), 2.9% (w/w), 3.0% (w/w), 3.1% (w/w), 3.2% (w/w), 3.3% (w/w), 3.4% (w/w), 3.5% (w/w), 3.6% (w/w), 3.7% (w/w), 3.8% (w/w), 3.9% (w/w), or 4.0% (w/w) of an emollient. In some embodiments, the topical composition disclosed herein can comprise 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1.0% (w/w), 1.1% (w/w), 1.2% (w/w), 1.3% (w/w), 1.4% (w/w), 1.5% (w/w), 1.6% (w/w), 1.7% (w/w), 1.8% (w/w), 1.9% (w/w), 2.0% (w/w), 2.1% (w/w), 2.2% (w/w), 2.3% (w/w), 2.4% (w/w), 2.5% (w/w), 2.6% (w/w), 2.7% (w/w), 2.8% (w/w), 2.9% (w/w), 3.0% (w/w), 3.1% (w/w), 3.2% (w/w), 3.3% (w/w), 3.4% (w/w), 3.5% (w/w), 3.6% (w/w), 3.7% (w/w). 3.8% (w/w), 3.9% (w/w), 4% (w/w), 5% (w/w), 6% (w/w), 7% (w/w), 8% (w/w), 9% (w/w), 10% (w/w), 20% (w/w) or 30% (w/w) of an emollient or a range defined by any two of the preceding values.

In some embodiments, a topical composition disclosed herein can comprise at least 0.1% (w/w), 0.2% (w/w), 0.3% (w/w), 0.4% w/w), 0.5% (w/w), 0.6% (w/w), 0.7% (w/w), 0.8% (w/w), 0.9% (w/w), 1.0% (w/w), 1.1% (w/w), 1.2% (w/w), 1.3% (w/w), 1.4% (w/w), 1.5% (w/w), 1.6% (w/w), 1.7% (w/w), 1.8% (w/w), 1.9% (w/w), 2.0% (w/w), 2.1% (w/w), 2.2% (w/w), 2.3% (w/w), 2.4% (w/w), 2.5% (w/w), 2.6% (w/w), 2.7% (w/w), 2.8% (w/w), 2.9% (w/w), 3.0% (w/w), 3.1% (w/w), 3.2% (w/w), 3.3% (w/w), 3.4% (w/w), 3.5% (w/w), 3.6% (w/w), 3.7% (w/w), 3.8% (w/w), 3.9% (w/w), or 4.0% (w/w) of glycerol. In some embodiments, the topical composition disclosed herein can comprise 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1.0% (w/w), 1.1% (w/w), 1.2% (w/w), 1.3% (w/w), 1.4% (w/w), 1.5% (w/w), 1.6% (w/w), 1.7% (w/w), 1.8% (w/w), 1.9% (w/w), 2.0% (w/w), 2.1% (w/w), 2.2% (w/w), 2.3% (w/w), 2.4% (w/w), 2.5% (w/w), 2.6% (w/w), 2.7% (w/w), 2.8% (w/w), 2.9% (w/w), 3.0% (w/w), 3.1% (w/w), 3.2% (w/w), 3.3% (w/w), 3.4% (w/w), 3.5% (w/w), 3.6% (w/w), 3.7% (w/w), 3.8% (w/w), 3.9% (w/w), 4% (w/w), 5% (w/w), 6% (w/w), 7% (w/w), 8% (w/w), 9% (w/w), 10% (w/w), 20% (w/w) or 30% (w/w) of glycerol or a range defined by any two of the preceding values.

In some embodiments, the excipients can include fatty acid triglycerides, namely the triglycerol esters of saturated and/or unsaturated, branched and/or unbranched alkanecarboxylic acids having a chain length of from 6 to 24 carbon atoms, in particular 6-10 carbon atoms. In a particular embodiment, the ester oil can be caprylic/capric triglyceride.

In some embodiments, a topical composition disclosed herein can comprise at least 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1.0% (w/w), 1.1% (w/w), or 1.2% (w/w) of a fatty acid triglyceride. In some embodiments, the topical composition disclosed herein can comprise 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1.0% (w/w), 1.1% (w/w), 1.2% (w/w), 1.5% (w/w), 2% (w/w), 3% (w/w), 4% (w/w), 5% (w/w), 6% (w/w), 7% (w/w), 8% (w/w), 9% (w/w), 10% (w/w), 20% (w/w) or 30% (w/w) of a fatty acid triglyceride or a range defined by any two of the preceding values.

In some embodiments, a topical composition disclosed herein can comprise at least 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1.0% (w/w), 1.1% (w/w), or 1.2% (w/w) of caprylic/capric triglyceride. In some embodiments, the topical composition disclosed herein can comprise 0.1% (w/w), 0.2% (w/w), 0.3% (w/w), 0.4% (w/w), 0.5% w/w), 0.6% (w/w), 0.7% (w/w), 0.8% (w/w), 0.9% (w/w), 1.0% (w/w), 1.1% (w/w), 1.2% w/w), 1.5% (w/w), 2% (w/w), 3% (w/w), 4% (w/w), 5% (w/w), 6% (w/w), 7% (w/w), 8% (w/w), 9% (w/w), 10% (w/w), 20% (w/w) or 30% (w/w) of caprylic/capric triglyceride or a range defined by any two of the preceding values.

In some embodiments, the excipients can include fatty acid triglycerides, namely the triglycerol esters of saturated and/or unsaturated, branched and/or unbranched alkanecarboxylic acids having a chain length of from 6 to 24 carbon atoms, in particular 6-10 carbon atoms. In a particular embodiment, the fatty acid triglyceride can be caprylic/capric triglyceride.

In some embodiments, the excipients can include fatty acid diglycerides, namely the diglycerol esters of saturated and/or unsaturated, branched and/or unbranched alkanecarboxylic acids having a chain length of from 6 to 2.4 carbon atoms, in particular 6-10 carbon atoms.

In some embodiments, the excipients can include an emulsifier. Suitable emulsifiers are disclosed in, for example, in McCutcheon's Detergents and Emulsifiers, North American Edition, pp. 317-324 (1986), and the ICI Handbook, pp. 1673-1686, which are incorporated herein by reference in their entirety. In some embodiments, the emulsifier can include glycerol monostearate.

In some embodiments, the compositions disclosed herein can comprise at least 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1% (w/w), 2% (w/w), 3% (w/w), 4% (w/w), 5% (w/w), 6% (w/w), 7% (w/w), 8% (w/w), 9% (w/w), 10% (w/w), 11% (w/w), 12% (w/w), 13% (w/w), 14% (w/w), 15% (w/w), 16% (w/w), 17% (w/w), 18% (w/w), 19% (w/w), 20% (w/w), 30% (w/w), or 40% (w/w) of an emulsifier. In some embodiments, the topical composition disclosed herein can comprise 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1% (w/w), 2% (w/w), 3% (w/w), 4% (w/w), 5% (w/w), 6% (w/w), 7% (w/w), 8% (w/w), 9% (w/w), 10% (w/w), 11% (w/w), 12% (w/w), 13% (w/w), 14% (w/w), 15% (w/w), 16% (w/w), 17% (w/w), 18% (w/w), 19% (w/w), 20% (w/w), 30% (w/w), or 40% (w/w) of an emulsifier or a range defined by any two of the preceding values. In some embodiments, the emulsifier can include one or more components, two or more components or three or more components.

In some embodiments, the composition disclosed herein can comprise at least 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1% (w/w). 2% (w/w), 3% (w/w), 4% (w/w), 5% (w/w), 6% (w/w), 7% (w/w), 8% (w/w), 9% (w/w), 10% (w/w), 11% (w/w), 12% (w/w), 13% (w/w), 14% (w/w), 15% (w/w), 16% (w/w), 17% (w/w), 18% (w/w), 19% (w/w), 20% (w/w), 30% (w/w), or 40% (w/w) of an emulsifier including glycerol monostearate, PEG-6 Stearate, Glycol stearate and PEG-32 stearate. In some embodiments, the topical composition disclosed herein can comprise 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1% (w/w), 2% (w/w), 3% (w/w), 4% (w/w), 5% (w/w), 6% (w/w), 7%(w/w), 8% (w/w), 9% (w/w), 10% (w/w), 11% (w/w), 12% (w/w), 13% (w/w), 14% (w/w), 15% (w/w), 16% (w/w), 17% (w/w), 18% (w/w), 19% (w/w), 20% (w/w), 30% (w/w), or 40% (w/w) of an emulsifier including glycerol monostearate, PEG-6 Stearate, Glycol stearate and PEG-32 stearate or a range defined by any two of the preceding values. In some embodiments, the can be a mixture of glycerol monostearate, PEG-6 Stearate, Glycol stearate and PEG-32 stearate.

In some embodiments, the excipients can include preservatives. In some embodiments, the preservatives can be selected from the group consisting of benzyl alcohol, methyl paraben, propyl paraben, DMDM hydantoin, methylchloroisothiaoline, methylisothiazolinone, imidazolidinyl urea phenoxyethanol, sodium benzoate and benzoic acid. In some embodiments, the preservatives can include phenoxyethanol, propyl paraben, and methyl paraben. In some embodiments, the preservatives can include benzalkonium chloride and/or poly(hexamethylenebiguanide) hydrochloride (PHMB).

In some embodiments, the composition disclosed herein can comprise at least 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1.0% (w/w), 1.1% (w/w), or 1.2% (w/w) of a preservative. In some embodiments, the topical composition disclosed herein can comprise 0.1% (w/w), 0.2% (w/w), 0.3% (w/w), 0.4% (w/w), 0.5% (w/w), 0.6% (w/w), 0.7% (w/W), 0.8% (w/w), 0.9% (w/w), 1.0% w/w), 1.1% (w/w), 1.2% (w/w), 1.5% (w/w), 2% (w/w), 3% (w/w), 4% (w/w), 5% (w/w), 6% (w/w), 7% (w/w), 8% (w/w), 9% (w/w), 10% (w/w), 20% (w/w) or 30% (w/w) of a preservative or a range defined by any two of the preceding values. In some embodiments, the preservative can include one or more components, two or more components or three or more components,

In some embodiments, the composition disclosed herein can comprise at least 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1.0% (w/w), 1.1% (w/w), or 1.2% (w/w) of a preservative including phenoxyethanol, propyl paraben, and methyl paraben. In some embodiments, the topical composition disclosed herein can comprise 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1.0% (w/w), 1.1% (w/w), 1.2% (w/w), 1.5% (w/w), 2% (w/w), 3% (w/w), 4% (w/w), 5% (w/w), 6% (w/w), 7% (w/w), 8% (w/w), 9% (w/w), 10% (w/w), 20% (w/w) or 30% (w/w) of a preservative including phenoxyethanol, propyl paraben, and methyl paraben or a range defined by any two of the preceding values.

In some embodiments, the composition may include colorants, deodorants, fragrances, perfumes, anti-foaming agents, lubricants, natural moisturizing agents, skin conditioning agents, skin protectants, skin benefit agents, solvents, solubilizing agents, suspending agents, wetting agents, humectants, propellants, dyes, pigments, and combinations thereof.

In some embodiments, the composition may include additional components added to enhance the odor, texture or color of the composition. For example, fragrances may be added to enhance odor. For example, emulsifiers or inert spheres may be added to enhance texture. For example, colorants may be added to enhance color.

In some embodiments, the composition may be applied to a body portion, such as a hand, foot, knee, elbow, and the like to treat pain and/or inflammation of the body portion. The composition may be applied by any suitable means, such as rubbing, spraying, rolling, wiping, and the like, and massaged into the body portion to be treated.

In some embodiments, the compounds as disclosed and described herein and/or topical compositions thereof can be used in combination therapy with at least one other agent. In some embodiments, a compound as disclosed and described herein and/or topical composition thereof is administered concurrently with the administration of another agent, which may be part of the same topical composition as the compound of the present invention or a different composition. In other embodiments, a topical composition of the present invention is administered prior or subsequent to administration of another agent.

In some embodiments the compositions described herein are incorporated into a patch or film for transdermal delivery. In some embodiments, such patches further comprise a porous or resorbable film, an active pharmaceutical agent, and optionally a transdermal carrier or penetration enhancer. Exemplary transdermal carriers include dimethylsulfoxide; 1-dodecylazacycloheptan-2-one or laurocapran; dimethylacetamide; dimethylformamide; lauric acid; myristic acid; cupric acid; caprylic acid; oleic acid; diethylene glycol; tetraethylene glycol; terpenes; essential oils of eucalyptus, chenopodium and ylang-ylang; dimethyl isosorbide; Oxazolidinones such as 4-decyloxazolidin-2-one; 2-pyrrolidone; N-methyl-2-pyrrolidone; urea; EDTA; Sodium Glycolate; polysorbates; sodium deoxycholate; polyethylene glycol; PLA/PLGA nanoparticles; polymer nanoparticles; block-copolymer nanoparticles, especially those comprising Pluronic®-type polyethylene oxide-block-polypropylene oxide copolymers; porous silica nanoparticles; metallic nanoparticles, especially those comprising gold, palladium, and iron; metal oxide nanoparticles, especially those comprising TiO₂ and Al₂O₃, short chain alcohols such as ethanol, propanol, and butanol; and oils such as mineral oil and coconut oil. In some embodiments the compositions described herein are incorporated into an adhesive for a transdermal patch. In some further embodiments, the compositions described herein are incorporated into a resorbable film. In some embodiments, the active pharmaceutical agent is contained within a separate reservoir layer. In some embodiments, the transdermal patch consists of a single layer. In some embodiments, the transdermal patch is constructed in multiple layers.

Provided herein are compositions useful for the treatment of skin conditions or for improving the appearance of the skin, comprising extracts from plants of the genus Cannabis. Said extracts may be obtained by liquid extraction, especially utilizing liquid C₁-C₈ alkane, liquid C₁-C₈ alkene, liquid helium, liquid hydrogen, liquid neon, liquid xenon, liquid argon, liquid carbon dioxide, liquid nitrogen, liquid oxygen, liquid butane, liquid butene, liquid isobutane, liquid propane, liquid isopropane, liquid methane, or liquid ethane, as carriers, where the choice of carrier gas may be made by one of ordinary skill in the art with respect to the needs imposed by specific processes, locations, and feedstocks. The extraction as described herein may comprise the steps of passing the carrier gas over a feedstock, followed by collection of the extract and removal of the carrier gas. Removal of the carrier gas may be accomplished by degassing the extract under ambient pressure, or under a controlled pressure regimen. The feedstock may comprise any part of the Cannabis plant, including the leaves, buds, trichotnes, stems, seeds, arils, flowers, or roots. In one embodiment, root matter is chopped and packed into a column, and isobutene or carbon dioxide is passed over the root matter. The resulting extract can be collected and degassed for further use.

It is well known in the art that extracts of the cannabis plant contain tetrahydrocannabinolic acid (THCA) and cannabidiolic acid (CBDA), in varying amounts. Conventionally, heat is applied to the extracts to convert these two aforementioned compounds into tetrahydrocannabinol (THC) and cannabidiol (CBD), respectively. Conventionally, THC and CBD are regarded as being the active ingredients of a cannabis extract. It is an object of the present disclosure to provide compositions wherein the extract is not heated, including compositions resulting from extractions carried out at low temperature so as to inhibit the conversion of THCA and CBDA into THC and CBD. It is a further object of the present disclosure to provide compositions in which CBDA is the predominant cannabinoid. The present disclosure further provides compositions in which the presence of THC is minimized. In a preferred embodiment, the extraction is carried out at low temperature using root material as a feedstock, based on the surprising finding that the root mass of plants of the genus Cannabis provides extracts that are unusually high in CBDA and unusually low in THC. In some embodiments, the mass ratio of THCA to THC is between 0.7 and 1.4, between 0.6 and 2, or between 0.5 and 3. In some embodiments, the mass ratio of THCA to THC is greater than 1, greater then 1.4, or greater than 2. In some embodiments, the mass ratio of CBDA to CBD is from 2 to 9, from 4 to 9, or from 7 to 9. In some embodiments, the mass ratio of CBDA to CBD is greater than 2, greater than 4, greater than 7, or greater than 9. In some embodiments, the mass ratio of THC to CBD is less than 0.2, less than 0.06, less than 0.02, or less than 0.005. In some embodiments, the mass ratio of THCA to CBDA is less than 2.5, less than 0.4, less than 0.3, less than 0.06 or less than 0.03. in some embodiments, CBDA is present in higher amounts than any one of the other identified cannabinoids. In some embodiments, the composition is substantially free of THC. In some embodiments, the composition is substantially free of THC and THCA. In some embodiments, the composition is substantially free of THC, THCA, and CBD. In some embodiments, the composition is substantially free of THC, THCA, or CBD. The compositions as described herein may also be constructed by the combination of purified, synthetics, or biosynthetic CBDA, or by the combination of purified, synthetic, or biosynthetic CBDA with purified, synthetic, or biosynthetic THC, THCA, or CBD, wherein the THC, THCA, or CBD, wherein the mass of THC, THCA, or CBD in the final composition is lower than the mass of CBDA.

In the compositions according to the present disclosure, CBDA-containing extracts may be used directly or may be incorporated into formulations utilizing the aforementioned carriers, emollients, and other excipients. In some embodiments according to the present disclosure, a formulation may comprise CBDA, whether or not CBD, THC, or THCA are present, in combination with one or more of the aforementioned carriers, emollients, and other excipients.

Provided herein is a treatment for skin conditions, including, but not limited to, atopic dermatitis, stasis dermatitis, toxic dermatitis or contact dermatitis, psoriasis, rosacea, pruritis, acne, scarring or keloid, zoster, herpetic lesion, seborrheic dermatitis, impetigo, ichthyosis vulgaris, lichen planus, actinic keratosis, insect bites, insect stings, allergenic effects, burns, scrapes, cuts, abrasions, dandruff, athlete's foot, or skin irritation as a result of contact with poison ivy, poison oak, poison sumac, stinging nettle or other poisonous plants, or any condition marked by discoloration, wrinkling, or discomfort of the skin wherein an effective amount of any of the compositions as described herein is administered to a subject in need thereof. Provided herein is also a method of treating scarring, discoloration, wrinkles, rosacea, or the like, wherein an effective amount of any of the compositions as described herein is administered to a subject in need thereof, resulting in the amelioration of discomfort, or in the alteration of aesthetic appearance due to the reduction in said scarring, discoloration, wrinkles, rosacea, or the like, both in human subjects and in non-human animals. Additionally, the compositions described herein may be used as or incorporated into cosmetic products for the improvement of, or with the intention of improving, the appearance of the skin or external features of the body. Said compositions may also he applied to non-human animals for the improvement of, or with the intention of improving, the appearance of the skin or external features of the body.

### EXAMPLES

### Example 1

Approximately 100 g. of plant matter derived from cannabis stems was ground and placed in a glass column. 300 mL of liquid butane was passed over the plant matter and the resulting fluid was collected without fractionation. The collected fluid was degassed for 12 hours under ventilation at 23° C. The resulting oil was maintained at 4° C. until further use. A sample of the resulting oil was subjected to analysis by GCMS and was found to contain 27.7% tetrahydrocannabinoic acid, 0.7% cannabidiolic acid, and 20.2% and 0.1% THC and CBD, respectively. This process was repeated using various other sources of plant matter including buds (“BHO”), leaves (“2A pulp”), and root matter (“Herman”). See Table 1.

TABLE 1

| | THC | CBD | | |
|---|---|---|---|---|
| Sample: | Content | Content | THCA Content | CBDA Content |
| BHO | 20.2% | 0.1% | 27.7% | 0.7% |
| 2A Pulp | 16.6% | 0.9% | 5.6% | 2.0% |
| 2B Essence | 0.0% | 0.0% | 0.0% | 0.0% |
| Herman | 9.7% | 1.8% | 8.1% | 17.5% |
| Combo with | 5.2% | 0.1% | 6.5% | 0.4% |
| Coconut | | | | |
| Combo/Pulp | 1.1% | 0.0% | 0.4% | 0.1% |
| with Coconut | | | | |

### Example 2

Approximately 1 g of a composition manufactured according to Example 1 (“Herman”, see Table 1) was applied to a visible scar on the neck of a human subject. Observations were made at 24, 48, and 72 hours. After 48 hours, visible reductions in the boundaries of the scar tissue had occurred, and the coloration of the scar tissue more closely resembled that of the surrounding skin.

### Example 3

Approximately 1 g of a composition manufactured according to Example 1 (“Herman”, see Table 1) was applied to the facial skin of an adult human female. Observations were made at 24, 48, and 72 hours. After 48 hours, visible reductions had occurred in the extent of facial wrinkling, with noticeable tightening of the skin and an observable more youthful overall appearance.

### Example 4

Approximately 1 g of a composition manufactured according to Example 1 (“Herman”, see Table 1) was applied to the facial skin of an adult female Pit Bull Dog suffering from extensive dermatitis and hair loss. Observations were made at 24, 48, and 72 hours, and again after 3 weeks. After 48 hours, visible reductions had occurred in the extent of irritation due to dermatitis. After 3 weeks, dermatitis was not observable and substantial regrowth of fur had occurred.

### Example 5

Approximately 100 g. of an isolated C. sativa root ball was ground and placed in a glass column. 300 mL of liquid butane was passed over the plant matter and the resulting fluid was collected without fractionation. The collected fluid was degassed for 12 hours under ventilation at 23° C. The resulting oil was maintained at 4° C. until further use. A sample of the resulting oil was subjected to analysis by GCMS and was found to contain 8.1% tetrahydrocannabinoic acid, 17.5% cannabidiolic acid, and 9.7% and 1.8% of THC and CBD, respectively. The root ball tissue yielded a significantly higher fraction of CBDA relative to other plant tissues. See Table 1 (“Herman”).

### Example 6

Approximately 1 g of a composition manufactured according to Example 1 (“Herman”, see Table 1) was applied to a visible mole on the neck of a female human subject. Observations were made at 24, 48, and 72 hours. After 48 hours, visible reductions in the boundaries of the mole had occurred, and the coloration of the mole more closely resembled that of the surrounding skin.

### Example 7

Approximately 1 g of a composition manufactured according to Example 1 (“2A Pulp”, see Table 1) was applied to the facial skin of an adult human female. Observations were made at 24, 48, and 72 hours. No change was observed, suggesting that high-THC formulations are ineffective in treating signs of skin aging.

The above-described embodiments have been provided by way of example, and the methods and compositions described herein are not limited to these examples. Multiple variations and modification to the disclosed embodiments will occur, to the extent not mutually exclusive, to those skilled in the art upon consideration of the foregoing description. Additionally, other combinations, omissions, substitutions and modifications will be apparent to the skilled artisan in view of the disclosure herein. Accordingly, the methods and compositions disclosed herein are not intended to be limited by the disclosed embodiments, but are to be defined by reference to the appended claims. Those skilled in the art will recognize, or be able to ascertain using no more than routine experimentation, many equivalents to the specific embodiments of the methods and compositions described herein. Such equivalents are intended to be encompassed by the following claims.

1. A composition comprising an effective amount of tetrahydrocannabinolic acid or cannabadiolic acid and further comprising a pharmaceutically or cosmetically acceptable excipient.

2-24. (canceled)

25. The composition of claim 1 wherein the pharmaceutically or cosmetically acceptable excipient comprises a carrier, diluent, binder, thickener, emulsifier, stabilizer, emollient, abrasive, oil, wax, colloid, humectant, or moisturizer.

26. The composition of claim 1 further comprising a second therapeutic agent.

27. The composition of claim 26, wherein the second therapeutic agent is selected from the group consisting of a vitamin, an analgesic, an antipyretic, a cooling agent, a warming agent, a collagenase, a sunscreen, an anesthetic, an antibiotic, an antiviral, an antifungal, a depilatory, a hair-growth enhancer, a steroid, and an antipruritic.

28. The composition of claim 1, wherein the tetrahydrocannabinolic acid or cannabadiolic acid t is produced by:
extracting from the plant matter of a plant of the genus Cannabis said tetrahydrocannabinolic Acid or cannabadiolic acid utilizing a carrier selected from the group consisting of a liquid C₁-C₈ alkane, liquid helium, liquid hydrogen, liquid neon, liquid xenon, liquid argon, liquid carbon dioxide, liquid nitrogen, and liquid oxygen or any combination of the aforementioned carriers; and
removing the carrier;
wherein the temperature of the composition does not rise above 37° C. prior to the addition of an excipient or additive.

29. The composition of claim 28 wherein the temperature of the composition does not rise above does not rise above 25° C. prior to the addition of an excipient or additive.

30. The composition of claim 28 wherein the temperature of the composition does not rise above 20° C., 15° C., 10° C., or 5° C. prior to the addition of an excipient or additive.

31. The composition of claim 28 wherein the temperature of the composition does not rise above 0° C. prior to the addition of an excipient or additive.

32. The composition of claim 28, wherein the plant matter comprises the stems, arils, roots, or seeds of a plant of the genus Cannabis.

33. The composition of claim 28, wherein the plant matter comprises the root ball from a plant of the genus Cannabis or a portion thereof.

34. The composition of claim 28, wherein the plant matter comprises the leaves or inflorescences from a plant of the genus Cannabis or a portion thereof.

35. The composition of claim 1, wherein the cannabidiolic acid is present in a larger quantity than the tetrahydrocannabinolic acid.

36. The composition of claim 1, wherein the composition is incorporated into a poultice configured for the treatment of burns, an anti-itch cream, an antibiotic ointment, an after-sun gel, an after-sun lotion, shaving product, deodorant, odorant, insect repellant, facial care products, body care product, cosmetic, soap product, lip product, a shaving cream, a shaving lotion, an after-shave lotion, a roll-on deodorant, a spray deodorant, an air freshener, a room deodorizer, a perfume, a cologne, a hand-soap, a facial soap, a lipstick, a lip balm, a lip gloss, a body lotion, or a shower gel.

37. A method of treating or ameliorating wrinkles, scars, atopic dermatitis, moles, skin discolorations, rosacea, insect bites, insect stings, allergenic effects, burns, scrapes, cuts, abrasions, psoriasis, dandruff, pruritus, itching, nasal complaints, sore throats, upper respiratory ailments, acne, athlete's foot, or skin irritation as a result of contact with poison ivy, poison oak, poison sumac, stinging nettle or other poisonous plants comprising identifying or selecting a subject in need thereof, and applying a composition comprising an effective amount of the composition of claim 1 to said subject.

38. The composition of claim 1, wherein the composition is formulated for veterinary use.

39. The method of claim 37, wherein the subject is a human.

40. The method of claim 37, wherein the subject is a non-human animal.

41. The method of claim 37, wherein the subject is a domestic animal.

42. The method of claim 37, wherein the subject is a dog, cat, hamster, gerbil, guinea pig, ferret, horse, donkey, mule, cow, domestic buffalo, camel, llama, alpaca, bison, yak, goat, sheep, pig, elk, deer, domestic antelope, or a non-human primate.

43. The method of claim 37, wherein the subject is a domestic dog.

44. The method of claim 37, wherein the composition is applied topically.

45. The method of claim 37, wherein the composition is administered subcutaneously.

46. The method of claim 37, wherein the composition is administered transdermally or by a patch, poultice, or microneedle.

47. The method of claim 37, wherein the composition is administered by iontophoresis.
